(19) United States
(12) Patent Application Publication
(10) Pub. No.: US 2024/0000524 A1
(43) Pub. Date: Jan. 4, 2024

(54) SYSTEM AND APPARATUS FOR MANIPULATING AN ELONGATED MEDICAL DEVICE IN A ROBOTIC CATHETER-BASED PROCEDURE SYSTEM

(71) Applicant: Corindus, Inc., Newton, MA (US)

(72) Inventors: Paul Gregory, Watertown, MA (US); Peter Falb, Hingham, MA (US); Wayne Boucher, Manchester, NH (US); Andrew Clark, Arlington, MA (US); Cameron Canale, Groton, MA (US); Steven J. Blacker, Framingham, MA (US)

(21) Appl. No.: 18/255,648

(22) PCT Filed: Jan. 14, 2021
(86) PCT No.: PCT/US2021/070042

§ 371 Date: Jun. 2, 2023

(51) Int. Cl. A61B 34/30; A61B 17/34

(52) U.S. Cl. CPC A61B 34/30; A61B 17/3462; A61B 2034/301; A61B 2034/303; A61M 25/0113

(57) ABSTRACT

A cassette for use in a robotic drive of a catheter-based procedure system includes a housing comprising a cradle configured to receive an elongated medical device having a longitudinal device axis, a connection mechanism coupled to the housing at a position below the longitudinal device axis, and a cover pivotably coupled to the housing using the connection mechanism.

### FIELD

The present invention relates generally to the field of robotic medical procedure systems and, in particular, to a system and apparatus for manipulating an elongated medical device in a robotic drive.

### BACKGROUND

Catheters and other elongated medical devices (EMDs) may be used for minimally invasive medical procedures for the diagnosis and treatment of diseases of various vascular systems, including neurovascular intervention (NVI) also known as neurointerventional surgery, percutaneous coronary intervention (PCI) and peripheral vascular intervention (PVI). These procedures typically involve navigating a guidewire through the vasculature, and via the guidewire advancing a catheter to deliver therapy. The catheterization procedure starts by gaining access into the appropriate vessel, such as an artery or vein, with an introducer sheath using standard percutaneous techniques. Through the introducer sheath, a sheath or guide catheter is then advanced over a diagnostic guidewire to a primary location such as an internal carotid artery for NVI, a coronary ostium for PCI, or a superficial femoral artery for PVI. A guidewire suitable for the vasculature is then navigated through the sheath or guide catheter to a target location in the vasculature. In certain situations, such as in tortuous anatomy, a support catheter or microcatheter is inserted over the guidewire to assist in navigating the guidewire. The physician or operator may use an imaging system (e.g., fluoroscope) to obtain a cine with a contrast injection and select a fixed frame for use as a roadmap to navigate the guidewire or catheter to the target location, for example, a lesion. Contrast-enhanced images are also obtained while the physician delivers the guidewire or catheter so that the physician can verify that the device is moving along the correct path to the target location. While observing the anatomy using fluoroscopy, the physician manipulates the proximal end of the guidewire or catheter to direct the distal tip into the appropriate vessels toward the lesion or target anatomical location and avoid advancing into side branches.

Robotic catheter-based procedure systems have been developed that may be used to aid a physician in performing catheterization procedures such as, for example, NVI, PCI and PVI. Examples of NVI procedures include coil embolization of aneurysms, liquid embolization of arteriovenous malformations and mechanical thrombectomy of large vessel occlusions in the setting of acute ischemic stroke. In an NVI procedure, the physician uses a robotic system to gain target lesion access by controlling the manipulation of a neurovascular guidewire and microcatheter to deliver the therapy to restore normal blood flow. Target access is enabled by the sheath or guide catheter but may also require an intermediate catheter for more distal territory or to provide adequate support for the microcatheter and guidewire. The distal tip of a guidewire is navigated into, or past, the lesion depending on the type of lesion and treatment. For treating aneurysms, the microcatheter is advanced into the lesion and the guidewire is removed and several embolization coils are deployed into the aneurysm through the microcatheter and used to block blood flow into the aneurysm. For treating arteriovenous malformations, a liquid embolic is injected into the malformation via a microcatheter. Mechanical thrombectomy to treat vessel occlusions can be achieved either through aspiration and/or use of a stent retriever. Depending on the location of the clot, aspiration is either done through an aspiration catheter, or through a microcatheter for smaller arteries. Once the aspiration catheter is at the lesion, negative pressure is applied to remove the clot through the catheter. Alternatively, the clot can be removed by deploying a stent retriever through the microcatheter. Once the clot has integrated into the stent retriever, the clot is retrieved by retracting the stent retriever and microcatheter (or intermediate catheter) into the guide catheter.

In PCI, the physician uses a robotic system to gain lesion access by manipulating a coronary guidewire to deliver the therapy and restore normal blood flow. The access is enabled by seating a guide catheter in a coronary ostium. The distal tip of the guidewire is navigated past the lesion and, for complex anatomies, a microcatheter may be used to provide adequate support for the guidewire. The blood flow is restored by delivering and deploying a stent or balloon at the lesion. The lesion may need preparation prior to stenting, by either delivering a balloon for pre-dilation of the lesion, or by performing atherectomy using, for example, a laser or rotational atherectomy catheter and a balloon over the guidewire. Diagnostic imaging and physiological measurements may be performed to determine appropriate therapy by using imaging catheters or fractional flow reserve (FFR) measurements.

In PVI, the physician uses a robotic system to deliver the therapy and restore blood flow with techniques similar to NVI. The distal tip of the guidewire is navigated past the lesion and a microcatheter may be used to provide adequate support for the guidewire for complex anatomies. The blood flow is restored by delivering and deploying a stent or balloon to the lesion. As with PCI, lesion preparation and diagnostic imaging may be used as well.

When support at the distal end of a catheter or guidewire is needed, for example, to navigate tortuous or calcified vasculature, to reach distal anatomical locations, or to cross hard lesions, an over-the-wire (OTW) catheter or coaxial system is used. An OTW catheter has a lumen for the guidewire that extends the full length of the catheter. This provides a relatively stable system because the guidewire is supported along the whole length. This system, however, has some disadvantages, including higher friction, and longer overall length compared to rapid-exchange catheters (see below). Typically to remove or exchange an OTW catheter while maintaining the position of the indwelling guidewire, the exposed length (outside of the patient) of guidewire must be longer than the OTW catheter. A 300 cm long guidewire is typically sufficient for this purpose and is often referred to as an exchange length guidewire. Due to the length of the guidewire, two operators are needed to remove or exchange an OTW catheter. This becomes even more challenging if a triple coaxial, known in the art as a tri-axial system, is used (quadruple coaxial catheters have also been known to be used). However, due to its stability, an OTW system is often used in NVI and PVI procedures. On the other hand, PCI procedures often use rapid exchange (or monorail) catheters. The guidewire lumen in a rapid exchange catheter runs only through a distal section of the catheter, called the monorail or rapid exchange (RX) section. With a RX system, the operator manipulates the interventional devices parallel to each other (as opposed to with an OTW system, in which the devices are manipulated in a serial configuration), and the exposed length of guidewire only needs to be slightly longer than the RX section of the catheter. A rapid exchange length guidewire is typically 180-200 cm long. Given the shorter length guidewire and monorail, RX catheters can be exchanged by a single operator. However, RX catheters are often inadequate when more distal support is needed.

### SUMMARY

In accordance with an embodiment, a cassette for use in a robotic drive of a catheter-based procedure system includes a housing comprising a cradle configured to receive an elongated medical device having a longitudinal device axis, a connection mechanism coupled to the housing at a position below the longitudinal device axis, and a cover pivotably coupled to the housing using the connection mechanism.

In accordance with another embodiment, a cassette for use in a robotic drive of a catheter-based procedure system includes a housing comprising a cradle configured to receive an elongated medical device having a longitudinal device axis, the housing having a distal end and a proximal end, a saddle positioned on the proximal end of the housing, the saddle configured to receive and restrain a hemostasis valve coupled to the elongated medical device, a connection mechanism coupled to the housing at a position below the longitudinal device axis and a cover pivotably coupled to the housing using the connection mechanism.

In accordance with another embodiment, a robotic drive system for driving one or more elongated medical devices includes a linear member, a device module coupled to the linear member, a distal support arm having a device support connection located distal to the device module, an introducer interface support coupled to the device support connection, the introducer interface support having a flexible tube, and an introducer sheath coupled to the introducer interface support.

### BRIEF DESCRIPTION OF THE DRAWINGS

The invention will become more fully understood from the following detailed description, taken in conjunction with the accompanying drawings, wherein the reference numerals refer to like parts in which:

FIG. 1 is a perspective view of an exemplary catheter-based procedure system in accordance with an embodiment;

FIG. 2 is a schematic block diagram of an exemplary catheter-based procedure system in accordance with an embodiment;

FIG. 3 is a perspective view of a robotic drive for a catheter-based procedure system in accordance with an embodiment;

FIG. 4 is a diagram illustrating an elongated medical device axis of manipulation and the introductory point into the patient;

FIGS. 5a and 5b are diagrams illustrating the effect of the thickness of a robotic drive on the loss of working length;

FIG. 6 is a diagram illustrating an exemplary orientation to minimize loss of working length;

FIG. 7 is a perspective view of a device module with a vertically mounted cassette in accordance with an embodiment;

FIG. 8 is a rear perspective view of a device module with a vertically mounted cassette in accordance with an embodiment;

FIG. 9 is a front view of a distal end of a device module with a vertically mounted cassette in accordance with an embodiment;

FIG. 10 is a front view of a distal end of a device module with a horizontally mounted cassette in accordance with an embodiment;

FIG. 11 is a front view of a cassette and elongated medical device in accordance with an embodiment;

FIG. 12 is a perspective view of a cassette configured for a vertical mount to a drive module in accordance with an embodiment;

FIG. 13 is a perspective view of an example elongated medical device in accordance with an embodiment;

FIG. 14 is a perspective view of a cassette with a cover in an open position and an elongated medical device in accordance with an embodiment;

FIG. 15 is a perspective view of a cassette with an elongated medical device positioned on a cover of the cassette in an open position before the elongated medical device is loaded in the cassette in accordance with an embodiment;

FIG. 16 is a front view of a cassette with a cover in an open position and an elongated medical device loaded in the cassette in accordance with an embodiment; and

FIG. 17 is a perspective view of an introducer interface support in accordance with an embodiment.

### DETAILED DESCRIPTION

The following definitions will be used herein. The term elongated medical device (EMD) refers to, but is not limited to, catheters (e.g. guide catheters, microcatheters, balloon/stent catheters), wire-based devices (guidewires, embolization coils, stent retrievers, etc.), and devices that have a combination of these. Wire-based EMD includes, but is not limited to, guidewires, microwires, a proximal pusher for embolization coils, stent retrievers, self-expanding stents, and flow divertors. Typically wire-based EMD's do not have a hub or handle at its proximal terminal end. In one embodiment the EMD is a catheter having a hub at a proximal end of the catheter and a flexible shaft extending from the hub toward the distal end of the catheter, wherein the shaft is more flexible than the hub. In one embodiment the catheter includes an intermediary portion that transitions between the hub and the shaft that has an intermediate flexibility that is less rigid than the hub and more rigid than the shaft. In one embodiment the intermediary portion is a strain relief.

The terms distal and proximal define relative locations of two different features. With respect to a robotic drive the terms distal and proximal are defined by the position of the robotic drive in its intended use relative to a patient. When used to define a relative position, the distal feature is the feature of the robotic drive that is closer to the patient than a proximal feature when the robotic drive is in its intended in-use position. Within a patient, any vasculature landmark further away along the path from the access point is considered more distal than a landmark closer to the access point, where the access point is the point at which the EMD enters the patient. Similarly, the proximal feature is the feature that is farther from the patient than the distal feature when the robotic drive in its intended in-use position. When used to define direction, the distal direction refers to a path on which something is moving or is aimed to move or along which something is pointing or facing from a proximal feature toward a distal feature and/or patient when the robotic drive is in its intended in-use position. The proximal direction is the opposite direction of the distal direction.

The term longitudinal axis of a member (e.g., an EMD or other element in the catheter-based procedure system) is the direction of orientation going from a proximal portion of the member to a distal portion of the member. By way of example, the longitudinal axis of a guidewire is the direction of orientation from a proximal portion of the guide wire toward a distal portion of the guidewire even though the guidewire may be non-linear in the relevant portion. The term axial movement of a member refers to translation of the member along the longitudinal axis of the member. When a distal end of an EMD is axially moved in a distal direction along its longitudinal axis into or further into the patient, the EMD is being advanced. When the distal end of an EMD is axially moved in a proximal direction along its longitudinal axis out of or further out of the patient, the EMD is being withdrawn. The term rotational movement of a member refers to change in angular orientation of the member about the local longitudinal axis of the member. Rotational movement of an EMD corresponds to clockwise or counterclockwise rotation of the EMD about its longitudinal axis due to an applied torque.

The term axial insertion refers to inserting a first member into a second member along the longitudinal axes of the second member. The term lateral insertion refers to inserting a first member into a second member along a direction in a plane perpendicular to the longitudinal axis of the second member. This can also be referred to as radial loading or side loading. The term pinch refers to releasably fixing an EMD to a member such that the EMD and member move together when the member moves. The term unpinch refers to releasing the EMD from a member such that the EMD and member move independently when the member moves. The term clamp refers to releasably fixing an EMD to a member such that the EMD's movement is constrained with respect to the member. The member can be fixed with respect to a global coordinate system or with respect to a local coordinate system. The term unclamp refers to releasing the EMD from the member such that the EMD can move independently.

The term grip refers to the application of a force or torque to an EMD by a drive mechanism that causes motion of the EMD without slip in at least one degree of freedom. The term ungrip refers to the release of the application of force or torque to the EMD by a drive mechanism such that the position of the EMD is no longer constrained. In one example, an EMD gripped between two tires will rotate about its longitudinal axis when the tires move longitudinally relative to one another. The rotational movement of the EMD is different than the movement of the two tires. The position of an EMD that is gripped is constrained by the drive mechanism. The term buckling refers to the tendency of a flexible EMD when under axial compression to bend away from the longitudinal axis or intended path along which it is being advanced. In one embodiment axial compression occurs in response to resistance from being navigated in the vasculature. The distance an EMD may be driven along its longitudinal axis without support before the EMD buckles is referred to herein as the device buckling distance. The device buckling distance is a function of the device's stiffness, geometry (including but not limited to diameter), and force being applied to the EMD. Buckling may cause the EMD to form an arcuate portion different than the intended path. Kinking is a case of buckling in which deformation of the EMD is non-elastic resulting in a permanent set.

The terms top, up, upper, and above refer to the general direction away from the direction of gravity and the terms bottom, down, lower, and below refer to the general direction in the direction of gravity. The term inwardly refers to the inner portion of a feature. The term outwardly refers to the outer portion of a feature. The term front refers to the side of the robotic drive (or an element of the robotic drive or other element of the catheter procedure system) that faces a bedside user and away from the positioning system, such as an articulating arm. The term rear refers to the side of the robotic drive (or an element of the robotic drive or other element of the catheter procedure system) that is closest to the positioning system, such as the articulating arm. The term sterile interface refers to an interface or boundary between a sterile and non-sterile unit. For example, a cassette may be a sterile interface between the robotic drive and at least one EMD. The term sterilizable unit refers to an apparatus that is capable of being sterilized (free from pathogenic microorganisms). This includes, but is not limited to, a cassette, consumable unit, drape, device adaptor, and sterilizable drive modules/units (which may include electromechanical components). Sterilizable Units may come into contact with the patient, other sterile devices, or anything else placed within the sterile field of a medical procedure.

The term on-device adaptor refers to sterile apparatus capable of releasably pinching an EMD to provide a driving interface. For example, the on-device adaptor is also known as an end-effector or EMD capturing device. In one non-limiting embodiment, the on-device adaptor is a collet that is operatively controlled robotically to rotate the EMD about its longitudinal axis, to pinch and/or unpinch the EMD to the collet, and/or to translate the EMD along its longitudinal axis. In one embodiment the on-device adaptor is a hub-drive mechanism such as a driven gear located on the hub of an EMD.

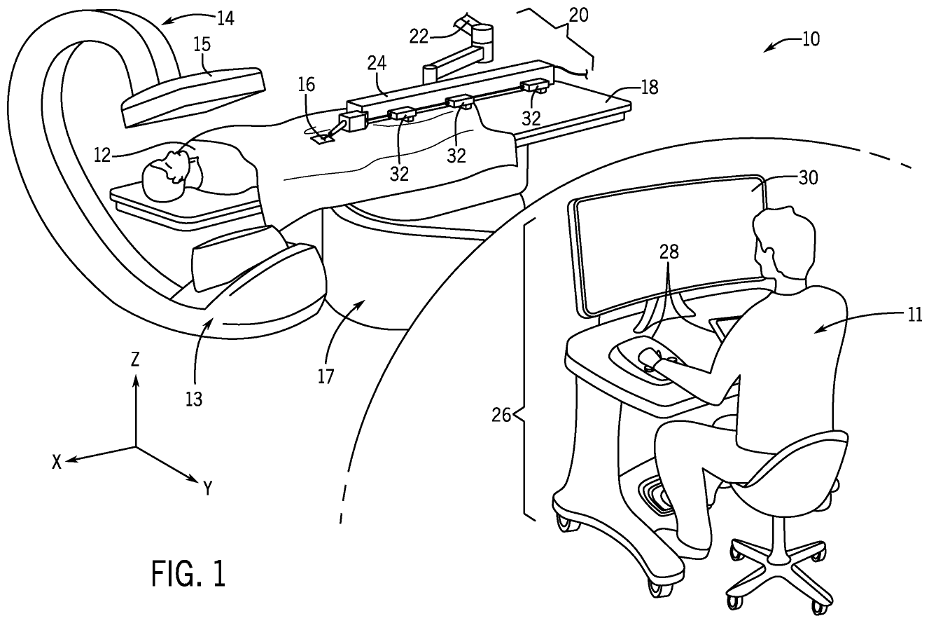

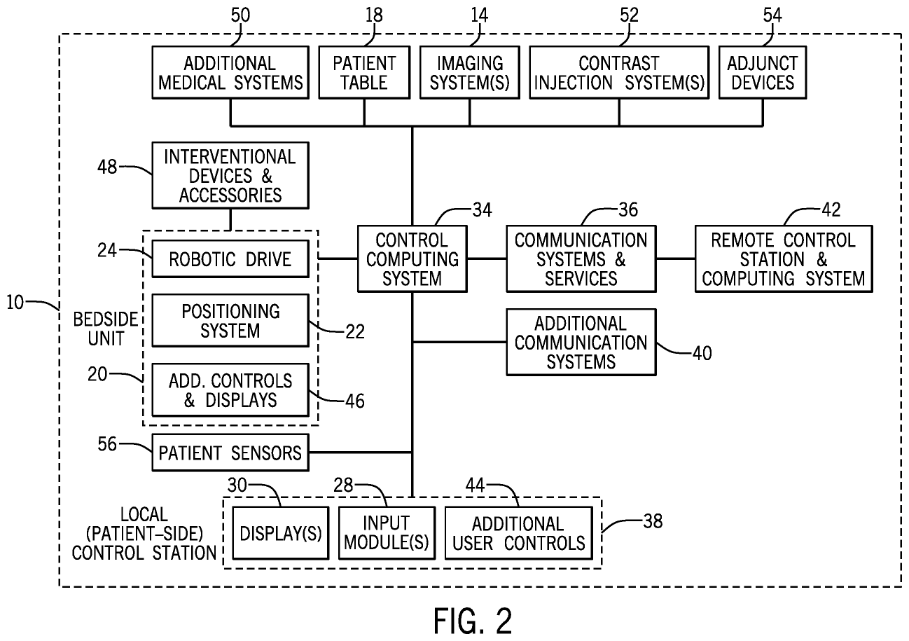

FIG. 1 is a perspective view of an exemplary catheter-based procedure system 10 in accordance with an embodiment. Catheter-based procedure system 10 may be used to perform catheter-based medical procedures, e.g., percutaneous intervention procedures such as a percutaneous coronary intervention (PCI) (e.g., to treat STEMI), a neurovascular interventional procedure (NVI) (e.g., to treat an emergent large vessel occlusion (ELVO)), peripheral vascular intervention procedures (PVI) (e.g., for critical limb ischemia (CLI), etc.). Catheter-based medical procedures may include diagnostic catheterization procedures during which one or more catheters or other elongated medical devices (EMDs) are used to aid in the diagnosis of a patient's disease. For example, during one embodiment of a catheter-based diagnostic procedure, a contrast media is injected onto one or more arteries through a catheter and an image of the patient's vasculature is taken. Catheter-based medical procedures may also include catheter-based therapeutic procedures (e.g., angioplasty, stent placement, treatment of peripheral vascular disease, clot removal, arterial venous malformation therapy, treatment of aneurysm, etc.) during which a catheter (or other EMD) is used to treat a disease. Therapeutic procedures may be enhanced by the inclusion of adjunct devices 54 (shown in FIG. 2) such as, for example, intravascular ultrasound (IVUS), optical coherence tomography (OCT), fractional flow reserve (FFR), etc. It should be noted, however, that one skilled in the art would recognize that certain specific percutaneous intervention devices or components (e.g., type of guidewire, type of catheter, etc.) may be selected based on the type of procedure that is to be performed. Catheter-based procedure system 10 can perform any number of catheter-based medical procedures with minor adjustments to accommodate the specific percutaneous intervention devices to be used in the procedure.

Catheter-based procedure system 10 includes, among other elements, a bedside unit 20 and a control station 26. Bedside unit 20 includes a robotic drive 24 and a positioning system 22 that are located adjacent to a patient 12. Patient 12 is supported on a patient table 18. The positioning system 22 is used to position and support the robotic drive 24. The positioning system 22 may be, for example, a robotic arm, an articulated arm, a holder, etc. The positioning system 22 may be attached at one end to, for example, a rail on the patient table 18, a base, or a cart. The other end of the positioning system 22 is attached to the robotic drive 24. The positioning system 22 may be moved out of the way (along with the robotic drive 24) to allow for the patient 12 to be placed on the patient table 18. Once the patient 12 is positioned on the patient table 18, the positioning system 22 may be used to situate or position the robotic drive 24 relative to the patient 12 for the procedure. In an embodiment, patient table 18 is operably supported by a pedestal 17, which is secured to the floor and/or earth. Patient table 18 is able to move with multiple degrees of freedom, for example, roll, pitch, and yaw, relative to the pedestal 17. Bedside unit 20 may also include controls and displays 46 (shown in FIG. 2). For example, controls and displays may be located on a housing of the robotic drive 24.

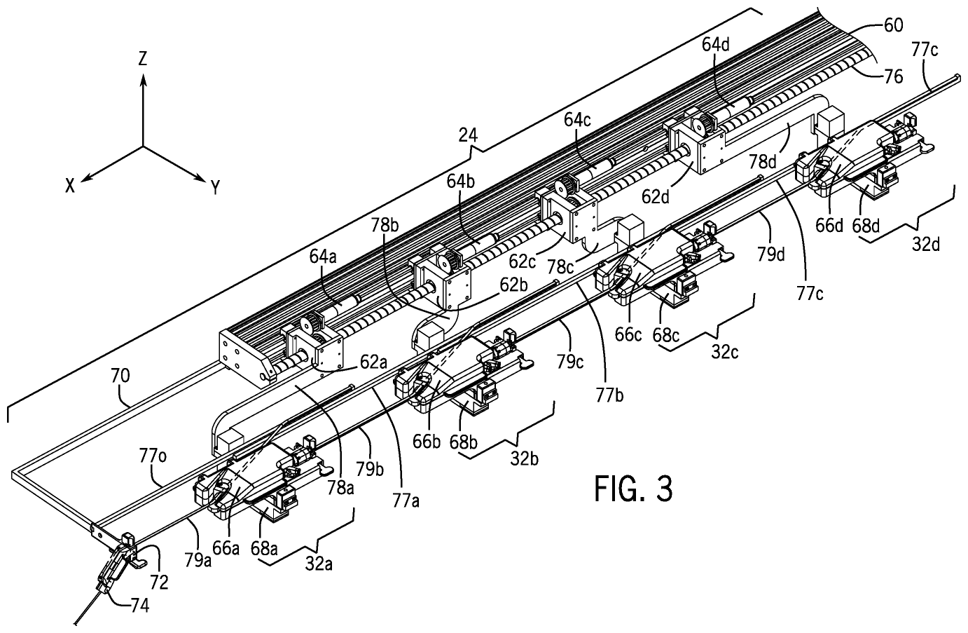

Generally, the robotic drive 24 may be equipped with the appropriate percutaneous interventional devices and accessories 48 (shown in FIG. 2) (e.g., guidewires, various types of catheters including balloon catheters, stent delivery systems, stent retrievers, embolization coils, liquid embolics, aspiration pumps, device to deliver contrast media, medicine, hemostasis valve adaptors, syringes, stopcocks, inflation device, etc.) to allow the user or operator 11 to perform a catheter-based medical procedure via a robotic system by operating various controls such as the controls and inputs located at the control station 26. Bedside unit 20, and in particular robotic drive 24, may include any number and/or combination of components to provide bedside unit 20 with the functionality described herein. A user or operator 11 at control station 26 is referred to as the control station user or control station operator and referred to herein as user or operator. A user or operator at bedside unit 20 is referred to as bedside unit user or bedside unit operator. The robotic drive 24 includes a plurality of device modules 32a-d mounted to a rail or linear member (shown in FIG. 3). The rail or linear member 60 guides and supports the device modules. Each of the device modules 32a-d may be used to drive an EMD such as a catheter or guidewire. For example, the robotic drive 24 may be used to automatically feed a guidewire into a diagnostic catheter and into a guide catheter in an artery of the patient 12. One or more devices, such as an EMD, enter the body (e.g., a vessel) of the patient 12 at an insertion point 16 via, for example, an introducer sheath.

Bedside unit 20 is in communication with control station 26, allowing signals generated by the user inputs of control station 26 to be transmitted wirelessly or via hardwire to bedside unit 20 to control various functions of bedside unit 20. As discussed below, control station 26 may include a control computing system 34 (shown in FIG. 2) or be coupled to the bedside unit 20 through a control computing system 34. Bedside unit 20 may also provide feedback signals (e.g., loads, speeds, operating conditions, warning signals, error codes, etc.) to control station 26, control computing system 34 (shown in FIG. 2), or both. Communication between the control computing system 34 and various components of the catheter-based procedure system 10 may be provided via a communication link that may be a wireless connection, cable connections, or any other means capable of allowing communication to occur between components. Control station 26 or other similar control system may be located either at a local site (e.g., local control station 38 shown in FIG. 2) or at a remote site (e.g., remote control station and computer system 42 shown in FIG. 2). Catheter procedure system 10 may be operated by a control station at the local site, a control station at a remote site, or both the local control station and the remote control station at the same time. At a local site, user or operator 11 and control station 26 are located in the same room or an adjacent room to the patient 12 and bedside unit 20. As used herein, a local site is the location of the bedside unit 20 and a patient 12 or subject (e.g., animal or cadaver) and the remote site is the location of a user or operator 11 and a control station 26 used to control the bedside unit 20 remotely. A control station 26 (and a control computing system) at a remote site and the bedside unit 20 and/or a control computing system at a local site may be in communication using communication systems and services 36 (shown in FIG. 2), for example, through the Internet. In an embodiment, the remote site and the local (patient) site are away from one another, for example, in different rooms in the same building, different buildings in the same city, different cities, or other different locations where the remote site does not have physical access to the bedside unit 20 and/or patient 12 at the local site.

Control station 26 generally includes one or more input modules 28 configured to receive user inputs to operate various components or systems of catheter-based procedure system 10. In the embodiment shown, control station 26 allows the user or operator 11 to control bedside unit 20 to perform a catheter-based medical procedure. For example, input modules 28 may be configured to cause bedside unit 20 to perform various tasks using percutaneous intervention devices (e.g., EMDs) interfaced with the robotic drive 24 (e.g., to advance, retract, or rotate a guidewire, advance, retract or rotate a catheter, inflate or deflate a balloon located on a catheter, position and/or deploy a stent, position and/or deploy a stent retriever, position and/or deploy a coil, inject contrast media into a catheter, inject liquid embolics into a catheter, inject medicine or saline into a catheter, aspirate on a catheter, or to perform any other function that may be performed as part of a catheter-based medical procedure). Robotic drive 24 includes various drive mechanisms to cause movement (e.g., axial and rotational movement) of the components of the bedside unit 20 including the percutaneous intervention devices.

In one embodiment, input modules 28 may include one or more touch screens, joysticks, scroll wheels, and/or buttons. In addition to input modules 28, the control station 26 may use additional user controls 44 (shown in FIG. 2) such as foot switches and microphones for voice commands, etc. Input modules 28 may be configured to advance, retract, or rotate various components and percutaneous intervention devices such as, for example, a guidewire, and one or more catheters or microcatheters. Buttons may include, for example, an emergency stop button, a multiplier button, device selection buttons and automated move buttons. When an emergency stop button is pushed, the power (e.g., electrical power) is shut off or removed to bedside unit 20. When in a speed control mode, a multiplier button acts to increase or decrease the speed at which the associated component is moved in response to a manipulation of input modules 28. When in a position control mode, a multiplier button changes the mapping between input distance and the output commanded distance. Device selection buttons allow the user or operator 11 to select which of the percutaneous intervention devices loaded into the robotic drive 24 are controlled by input modules 28. Automated move buttons are used to enable algorithmic movements that the catheter-based procedure system 10 may perform on a percutaneous intervention device without direct command from the user or operator 11. In one embodiment, input modules 28 may include one or more controls or icons (not shown) displayed on a touch screen (that may or may not be part of a display 30), that, when activated, causes operation of a component of the catheter-based procedure system 10. Input modules 28 may also include a balloon or stent control that is configured to inflate or deflate a balloon and/or deploy a stent. Each of the input modules 28 may include one or more buttons, scroll wheels, joysticks, touch screen, etc. that may be used to control the particular component or components to which the control is dedicated. In addition, one or more touch screens may display one or more icons (not shown) related to various portions of input modules 28 or to various components of catheter-based procedure system 10.

Control station 26 may include a display 30. In other embodiments, the control station 26 may include two or more displays 30. Display 30 may be configured to display information or patient specific data to the user or operator 11 located at control station 26. For example, display 30 may be configured to display image data (e.g., X-ray images, MRI images, CT images, ultrasound images, etc.), hemodynamic data (e.g., blood pressure, heart rate, etc.), patient record information (e.g., medical history, age, weight, etc.), lesion or treatment assessment data (e.g., IVUS, OCT, FFR, etc.). In addition, display 30 may be configured to display procedure specific information (e.g., procedural checklist, recommendations, duration of procedure, catheter or guidewire position, volume of medicine or contrast agent delivered, etc.). Further, display 30 may be configured to display information to provide the functionalities associated with control computing system 34 (shown in FIG. 2). Display 30 may include touch screen capabilities to provide some of the user input capabilities of the system.

Catheter-based procedure system 10 also includes an imaging system 14. Imaging system 14 may be any medical imaging system that may be used in conjunction with a catheter based medical procedure (e.g., non-digital X-ray, digital X-ray, CT, MRI, ultrasound, etc.). In an exemplary embodiment, imaging system 14 is a digital X-ray imaging device that is in communication with control station 26. In one embodiment, imaging system 14 may include a C-arm (shown in FIG. 1) that allows imaging system 14 to partially or completely rotate around patient 12 in order to obtain images at different angular positions relative to patient 12 (e.g., sagittal views, caudal views, anterior-posterior views, etc.). In one embodiment imaging system 14 is a fluoroscopy system including a C-arm having an X-ray source 13 and a detector 15, also known as an image intensifier.

Imaging system 14 may be configured to take X-ray images of the appropriate area of patient 12 during a procedure. For example, imaging system 14 may be configured to take one or more X-ray images of the head to diagnose a neurovascular condition. Imaging system 14 may also be configured to take one or more X-ray images (e.g., real time images) during a catheter-based medical procedure to assist the user or operator 11 of control station 26 to properly position a guidewire, guide catheter, microcatheter, stent retriever, coil, stent, balloon, etc. during the procedure. The image or images may be displayed on display 30. For example, images may be displayed on display 30 to allow the user or operator 11 to accurately move a guide catheter or guidewire into the proper position.

In order to clarify directions, a rectangular coordinate system is introduced with X, Y, and Z axes. The positive X axis is oriented in a longitudinal (axial) distal direction, that is, in the direction from the proximal end to the distal end, stated another way from the proximal to distal direction. The Y and Z axes are in a transverse plane to the X axis, with the positive Z axis oriented up, that is, in the direction opposite of gravity, and the Y axis is automatically determined by right-hand rule.

FIG. 2 is a block diagram of catheter-based procedure system 10 in accordance with an exemplary embodiment. Catheter-procedure system 10 may include a control computing system 34. Control computing system 34 may physically be, for example, part of control station 26 (shown in FIG. 1). Control computing system 34 may generally be an electronic control unit suitable to provide catheter-based procedure system 10 with the various functionalities described herein. For example, control computing system 34 may be an embedded system, a dedicated circuit, a general-purpose system programmed with the functionality described herein, etc. Control computing system 34 is in communication with bedside unit 20, communications systems and services 36 (e.g., Internet, firewalls, cloud services, session managers, a hospital network, etc.), a local control station 38, additional communications systems 40 (e.g., a telepresence system), a remote control station and computing system 42, and patient sensors 56 (e.g., electrocardiogram (ECG) devices, electroencephalogram (EEG) devices, blood pressure monitors, temperature monitors, heart rate monitors, respiratory monitors, etc.). The control computing system is also in communication with imaging system 14, patient table 18, additional medical systems 50, contrast injection systems 52 and adjunct devices 54 (e.g., IVUS, OCT, FFR, etc.). The bedside unit 20 includes a robotic drive 24, a positioning system 22 and may include additional controls and displays 46. As mentioned above, the additional controls and displays may be located on a housing of the robotic drive 24. Interventional devices and accessories 48 (e.g., guidewires, catheters, etc.) interface to the bedside system 20. In an embodiment, interventional devices and accessories 48 may include specialized devices (e.g., IVUS catheter, OCT catheter, FFR wire, diagnostic catheter for contrast, etc.) which interface to their respective adjunct devices 54, namely, an IVUS system, an OCT system, and FFR system, etc.

In various embodiments, control computing system 34 is configured to generate control signals based on the user's interaction with input modules 28 (e.g., of a control station 26 (shown in FIG. 1) such as a local control station 38 or a remote control station 42) and/or based on information accessible to control computing system 34 such that a medical procedure may be performed using catheter-based procedure system 10. The local control station 38 includes one or more displays 30, one or more input modules 28, and additional user controls 44. The remote control station and computing system 42 may include similar components to the local control station 38. The remote 42 and local 38 control stations can be different and tailored based on their required functionalities. The additional user controls 44 may include, for example, one or more foot input controls. The foot input control may be configured to allow the user to select functions of the imaging system 14 such as turning on and off the X-ray and scrolling through different stored images. In another embodiment, a foot input device may be configured to allow the user to select which devices are mapped to scroll wheels included in input modules 28. Additional communication systems 40 (e.g., audio conference, video conference, telepresence, etc.) may be employed to help the operator interact with the patient, medical staff (e.g., angio-suite staff), and/or equipment in the vicinity of the bedside.

Catheter-based procedure system 10 may be connected or configured to include any other systems and/or devices not explicitly shown. For example, catheter-based procedure system 10 may include image processing engines, data storage and archive systems, automatic balloon and/or stent inflation systems, medicine injection systems, medicine tracking and/or logging systems, user logs, encryption systems, systems to restrict access or use of catheter-based procedure system 10, etc.

As mentioned, control computing system 34 is in communication with bedside unit 20 which includes a robotic drive 24, a positioning system 22 and may include additional controls and displays 46, and may provide control signals to the bedside unit 20 to control the operation of the motors and drive mechanisms used to drive the percutaneous intervention devices (e.g., guidewire, catheter, etc.). The various drive mechanisms may be provided as part of a robotic drive 24. FIG. 3 is a perspective view of a robotic drive for a catheter-based procedure system 10 in accordance with an embodiment. In FIG. 3, a robotic drive 24 includes multiple device modules 32a-d coupled to a linear member 60. Each device module 32a-d is coupled to the linear member 60 via a stage 62a-d moveably mounted to the linear member 60. A device module 32a-d may be connected to a stage 62a-d using a connector such as an offset bracket 78a-d. In another embodiment, the device module 32a-d is directly mounted to the stage 62a-d. Each stage 62a-d may be independently actuated to move linearly along the linear member 60. Accordingly, each stage 62a-d (and the corresponding device module 32a-d coupled to the stage 62a-d) may independently move relative to each other and the linear member 60. A drive mechanism is used to actuate each stage 62a-d. In the embodiment shown in FIG. 3, the drive mechanism includes independent stage translation motors 64a-d coupled to each stage 62a-d and a stage drive mechanism 76, for example, a lead screw via a rotating nut, a rack via a pinion, a belt via a pinion or pulley, a chain via a sprocket, or the stage translation motors 64a-d may be linear motors themselves. In some embodiments, the stage drive mechanism 76 may be a combination of these mechanisms, for example, each stage 62a-d could employ a different type of stage drive mechanism. In an embodiment where the stage drive mechanism is a lead screw and rotating nut, the lead screw may be rotated and each stage 62a-d may engage and disengage from the lead screw to move, e.g., to advance or retract. In the embodiment shown in FIG. 3, the stages 62a-d and device modules 32a-d are in a serial drive configuration.

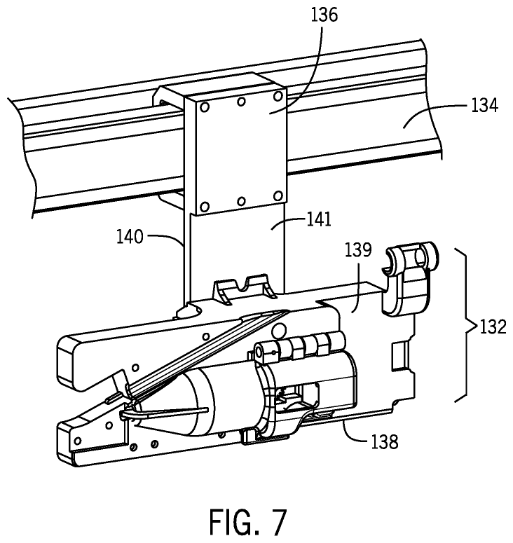

Each device module 32a-d includes a drive module 68a-d and a cassette 66a-d mounted on and coupled to the drive module 68a-d. In the embodiment shown in FIG. 3, each cassette 66a-d is mounted to the drive module 68a-d in an orientation such that the cassette 66a-d is mounted on a drive module 68a-d by moving the cassette 66a-d in a vertical direction down onto the drive module 66a-d. A top face or side of the cassette 66a-d is parallel to a top face or side (i.e., a mounting surface) of the drive module 68a-d when the cassette 66a-d is mounted on the drive module 68a-d. As used herein, the mounting orientation shown in FIG. 3 is referred to as a horizontal orientation. In other embodiments, each cassette 66a-d may be mounted to the drive module 68a-d in other mounting orientations. Various mounting orientations are described further below with respect to FIGS. 7-10. Each cassette 66a-d is configured to interface with and support a proximal portion of an EMD (not shown). In addition, each cassette 66a-d may include elements to provide one or more degrees of freedom in addition to the linear motion provided by the actuation of the corresponding stage 62a-d to move linearly along the linear member 60. For example, the cassette 66a-d may include elements that may be used to rotate the EMD when the cassette is coupled to the drive module 68a-d. Each drive module 68a-d includes at least one coupler to provide a drive interface to the mechanisms in each cassette 66a-d to provide the additional degree of freedom. Each cassette 66a-d also includes a channel in which a device support 79a-d is positioned, and each device support 79a-d is used to prevent an EMD from buckling. A support arm 77a, 77b, and 77c is attached to each device module 32a, 32b, and 32c, respectively, to provide a fixed point for support of a proximal end of the device supports 79b, 79c, and 79d, respectively. The robotic drive 24 may also include a device support connection 72 connected to a device support 79, a distal support arm 70 and a support arm 77₀. Support arm 77₀ is used to provide a fixed point for support of the proximal end of the distal most device support 79a housed in the distal most device module 32a. In addition, an introducer interface support (redirector) 74 may be connected to the device support connection 72 and an EMD (e.g., an introducer sheath). The configuration of robotic drive 24 has the benefit of reducing volume and weight of the drive robotic drive 24 by using actuators on a single linear member.

To prevent contaminating the patient with pathogens, healthcare staff use aseptic technique in a room housing the bedside unit 20 and the patient 12 or subject (shown in FIG. 1). A room housing the bedside unit 20 and patient 12 may be, for example, a cath lab or an angio suite. Aseptic technique consists of using sterile barriers, sterile equipment, proper patient preparation, environmental controls and contact guidelines. Accordingly, all EMDs and interventional accessories are sterilized and can only be in contact with either sterile barriers or sterile equipment. In an embodiment, a sterile drape (not shown) is placed over the non-sterile robotic drive 24. Each cassette 66a-d is sterilized and acts as a sterile interface between the draped robotic drive 24 and at least one EMD. Each cassette 66a-d can be designed to be sterile for single use or to be re-sterilized in whole or part so that the cassette 66a-d or its components can be used in multiple procedures.

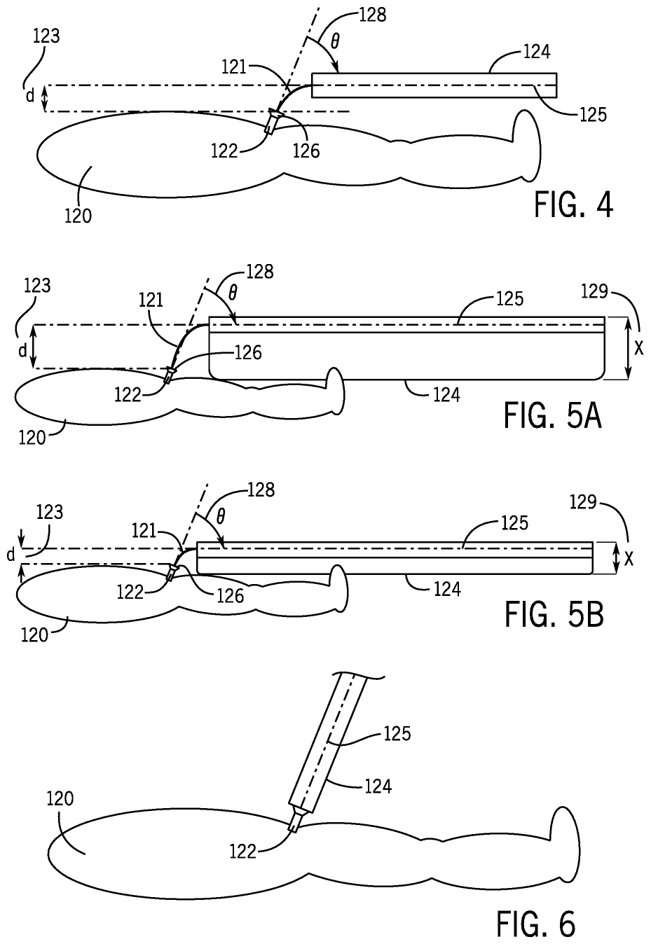

As shown in FIG. 1, one or more EMDs may enter the body of a patient (e.g., a vessel) at an insertion point 16 using, for example, an introducer and introducer sheath. The introducer sheath typically orients at an angle, usually less than 45 degrees, to the axis of the vessel in a patient 120 (shown in FIGS. 4-6). Any height difference between where the EMD enters the body (the introducer sheath's proximal opening 126 shown in FIG. 4) and the longitudinal drive axis of the robotic drive 124 will directly affect the working length for the elongated medical device. The more an elongated medical device needs to compensate for differences in displacement and angle, the less the elongated medical device will be able to enter the body when the robotic drive is at its maximum distal (forward) position. It is beneficial to have a robotic drive that is at the same height and angle as the introducer sheath. FIG. 4 is a diagram illustrating an elongated medical device axis of manipulation and the introductory point into the patient. FIG. 4 shows a height difference (d) 123 between the proximal end 126 of the introducer sheath 122 and the longitudinal device axis and an angular difference (θ) 128 between the introducer sheath 122 and the longitudinal device axis 125 of the robotic drive 124. The elongated medical device 121 is constrained on each axis and creates a curve with tangentially aligned end points. The length of this curve represents a length of the elongated medical device 121 that cannot be driven any further forward by the robotic drive 124 and cannot enter the introducer sheath 122 due to the misalignment. A higher angle (θ) 128 also leads to higher device friction. In general, lower angular misalignment (θ) 128, and linear misalignment d 123 can lead to reduced friction and reduced loss of working length. While FIG. 4 illustrates a simplified example illustrating one linear and one rotational offset, it should be understood that this problem occurs in three dimensions, namely, three linear offsets and three rotational offsets. The thickness of the robotic drive 124 also plays a role in determining the location of the longitudinal device axis 125 relative to the introducer sheath 122.

FIGS. 5a and 5b are diagrams illustrating the effect of the thickness of a drive module, or robotic drive as a whole, on the loss of working length. FIG. 5a shows the location of the longitudinal device axis 125 of a robotic drive 124 relative to the introducer sheath 122, indicated by d 123, when the robotic drive 124 is thick as shown by the distance (X) 129 between an upper surface and a bottom surface of the robotic drive 124. FIG. 5b shows the location of the longitudinal device axis 125 of a robotic drive 124 relative to the introducer sheath 122, indicated by a shorter d 123, ‘when the robotic drive 124 is shallow as shown by the distance (X) 129 between an upper surface and a bottom surface of the robotic drive 124. Reducing the thickness of the robotic drive 124 to get close to the patient and introducer sheath reduces the distance 123 between introducer sheath axis and device axis and reduces the loss of working length of the elongated medical device. FIG. 6 is a diagram illustrating an exemplary orientation to minimize loss of working length. In FIG. 6, the robotic drive is positioned to align the longitudinal device axis 125 of the robotic drive 124 to that of the introducer sheath 122. This eliminates loss of working length due to angular and linear misalignment of the elongated medical device. However, this position for the robotic drive 124 may not be practical due to the length and size of the robotic drive 124. Orienting a robotic drive at a sharp angle also affects the usability by making it difficult to load and unload elongated medical devices, and adjust and handle the robotic drive.

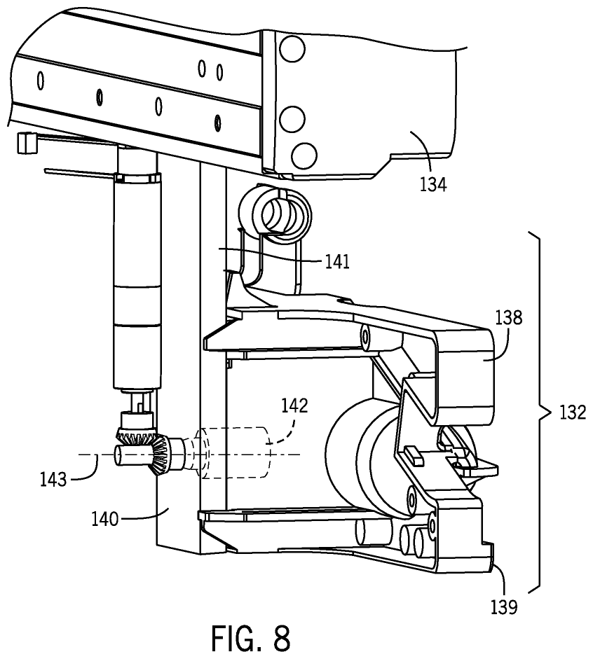

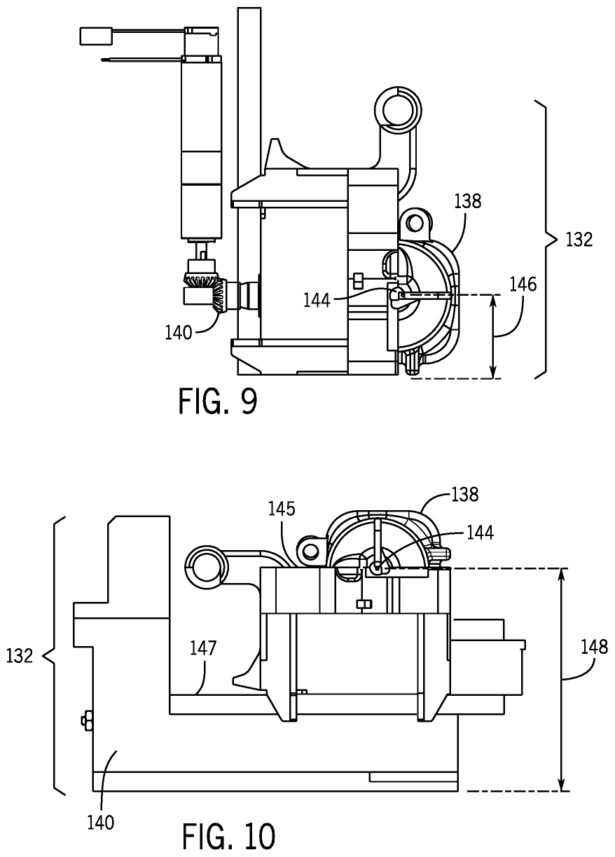

To reduce the distance between the robotic drive and the patient and the distance between the longitudinal device axis of the robotic drive and the introducer sheath, the cassette 66a-d of a device module 32 (shown in FIG. 3) may be mounted to the drive module 68a-d in a an orientation such that the cassette 66a-d is mounted on a drive module 68a-d by moving the cassette 66a-d in a horizontal direction onto the drive module 66a-d. FIG. 7 is a perspective view of a device module with a vertically mounted cassette in accordance with an embodiment and FIG. 8 is a rear perspective view of a device module with a vertically mounted cassette in accordance with an embodiment. In FIGS. 7 and 8, a device module 132 includes a cassette 138 that is mounted to a drive nodule 140 such that front face or side 139 of the cassette 138 is parallel to a front face or side 141 (i.e., a mounting surface) of the drive module 140. As used herein, the mounting orientation shown in FIGS. 7 and 8 is referred to as a vertical orientation. The device module 132 is connected to a stage 136 that is moveably mounted to a rail or linear member 134. The drive module 140 includes a coupler 142 that is used to provide a power interface to the cassette 138 to, for example, rotate an elongated medical device (not shown) positioned in the cassette. The coupler 142 rotates about an axis 143. As mentioned, the cassette 138 is mounted to the drive module 140 by moving the cassette 138 in a horizontal direction onto the mounting surface 141 so that the cassette is coupled to coupler 142 of the drive module 140. By mounting the cassette 138 vertically, the drive module 140 that the cassette 138 attaches to located off to the side and no longer positioned between the cassette 138 and the patient. FIG. 9 is a front view of a distal end of a device module with a vertically mounted cassette in accordance with an embodiment. In FIG. 9, a distance 146 between the device axis of the elongated medical device 144 and the bottom surface of the device module 132 is shown. The vertical mounting orientation of the cassette 138 eliminates the need for the drive module 140 to be placed under the device axis and between the elongated medical device 144 and the patient. Rather, only a portion of the cassette 138 is positioned between the elongated medical device 138 and the patient. Vertically mounting the cassette 138 also reduces the distance 146 between the elongated medical device and bottom surface of the device module 132 which allows the robotic drive to get closer to the patient and reduces loss of working length in an elongated medical device. By comparison, FIG. 10 is a front view of a distal end of a device module with a horizontally mounted cassette in accordance with an embodiment. In FIG. 10, a device module 132 is shown where the cassette 138 is horzontally mounted to a drive module 140. A top face or side 145 of the cassette 138 is parallel to a top face or side 147 (i.e., a mounting surface) of the drive module 140 when the cassette 138 is mounted on the drive module 140. The drive module 140 is under or below the cassette 138 and increases the distance 148 between the device axis of the elongated medical device 144 and the bottom surface of the device module 132. This can prevent the device axis from being as close to the introducer (not shown) as possible. A drive module 140 positioned under the cassette 138 may also interfere with the patient. In various other embodiment, a cassette may be mounted to the drive module at any angle. In yet another embodiment, the cassette may be mounted horizontally on an underside of the drive module to eliminate the need for a drive module between the device axis and the patient.

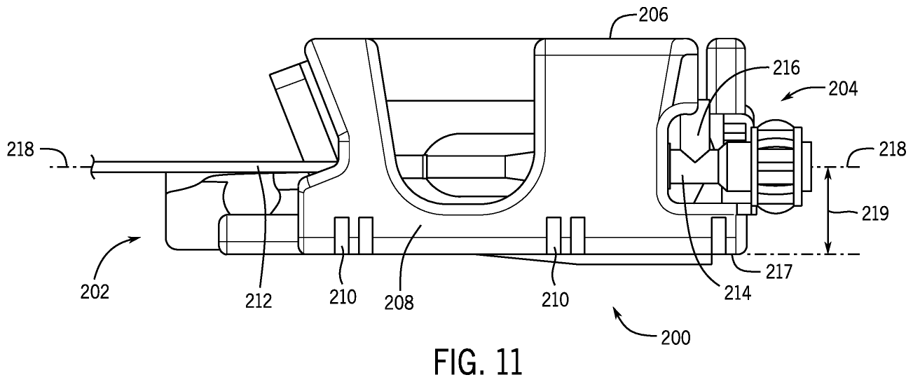

FIG. 11 is a front view of a cassette and elongated medical device in accordance with an embodiment. Cassette 200 is configured for a vertical mount to a drive module and includes features that enable the cassette 200 to be vertically mounted to a drive module (e.g., mounted in a vertical orientation as described above with respect to FIGS. 7-9) in a robotic drive. The cassette and drive module form a device module as described above with respect to FIG. 3. Cassette 200 has a distal end 202, a proximal end 204 and a longitudinal device axis 218 that is associated with and defined by an elongated medical device (EMD) 212 positioned in the cassette housing 206. In an embodiment, the longitudinal device axis 218 is below or lower than a central axis of the cassette 200 to bring the longitudinal device axis 218 closer to the patient. A distance 219 between the longitudinal device axis 218 and a bottom 217 of the device module (as defined by the cassette 200) can be reduced because the cassette 200 and drive module (not shown) are mounted vertically. Advantageously, in a vertical mount the drive module is not under the device axis and between the device axis and the patient. Accordingly, the longitudinal device axis 218 can get close to a patient, in particular, it is desirable to have the longitudinal device axis of the most distal device module (i.e., the device module closest to the patient along the linear member 60 (shown in FIG. 3) be as close to the patient as possible. In an embodiment, the cassette 200 is configured to minimize the distance 219. In an embodiment, the EMD 212 is a catheter. The catheter 212 is coupled to a hemostasis valve (e.g., a rotating hemostasis valve (RHV)) 214 which is also positioned in the cassette housing 206. The hemostasis valve 214 includes a side port 216 that may be connected to a tube (not shown) to facilitate the flow of a fluid (e.g., saline) to and from the hemostasis valve 214 and the catheter 212. Cassette 200 also includes a cover 208 that is connected to the cassette housing 206 using a connection mechanism 210 (e.g., a hinge). The connection mechanism 210 is located at a position below the longitudinal device axis 218. In FIG. 11, the cover 208 is in a closed position. The connection mechanism 210 enables the cover 208 to be moved from the closed position to an open position.

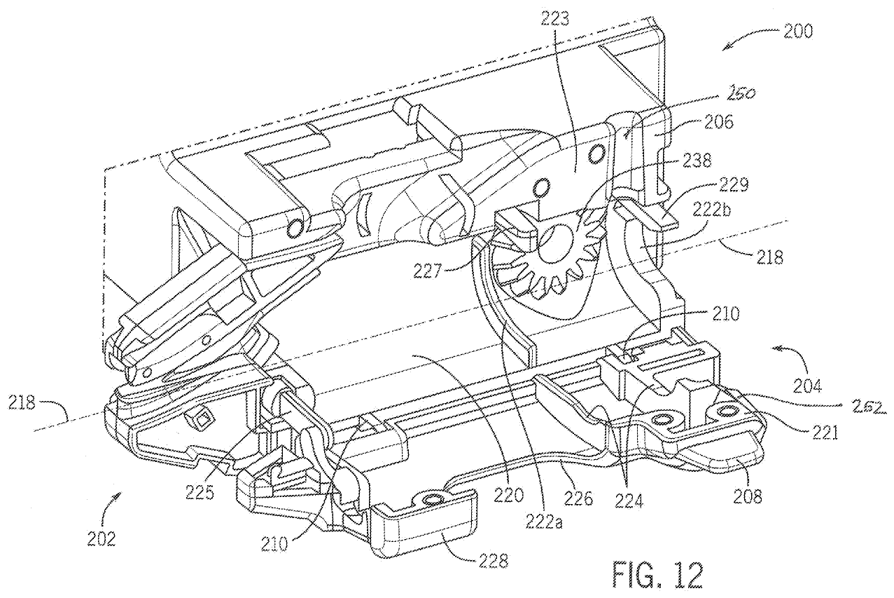

FIG. 12 is a perspective view of a cassette configured for a vertical mount to a drive module in accordance with an embodiment. In FIG. 12, a cover 208 connected to a housing 206 of a cassette 200 is in an open position. As mentioned above, the cover 208 may be attached to the cassette housing 206 with a connection mechanism 210 (e.g., a hinge). The connection mechanism 210 is located at a position below a longitudinal device axis 218 of the cassette 200. When the cover 208 is in the open position, a plane defined by an inner surface 221 of the cover 208 is substantially perpendicular to a plane defined by a front surface 223 of the cassette housing 206 and a front surface (e.g., front face or side 141 shown in FIG. 7) of a device module (not shown) to which the cassette 200 may be mounted in a vertical orientation. Accordingly, the cover 208 is in a horizontal orientation in the open position. In another embodiment, the cover 208 may be at an angle that allows the outer edge 228 of the cover 208 to be at a position that is lower than a horizontal orientation. A mechanical stop 225 is coupled to the cassette housing 206 and the cover 208 and is used to hold the cover 208 in the substantially horizontal orientation when the cover 208 is in an open position. In an embodiment, the mechanical stop 225 is coupled to the cassette housing 206 and the cover 208 and the mechanical stop 225 is used to hold the cover 208 at an angle below the horizontal when the cover 208 is in an open position. In a closed position (shown in FIG. 11), the plane defined by the inner surface 221 of the cover 208 is substantially parallel to the plane defined by the front surface 223 of the cassette housing 206 so that the cover 208 is in a vertical orientation. The cover 208 and/or the cassette housing 206 may include a mechanical locking feature or a magnet to hold the cover 206 in a closed position.

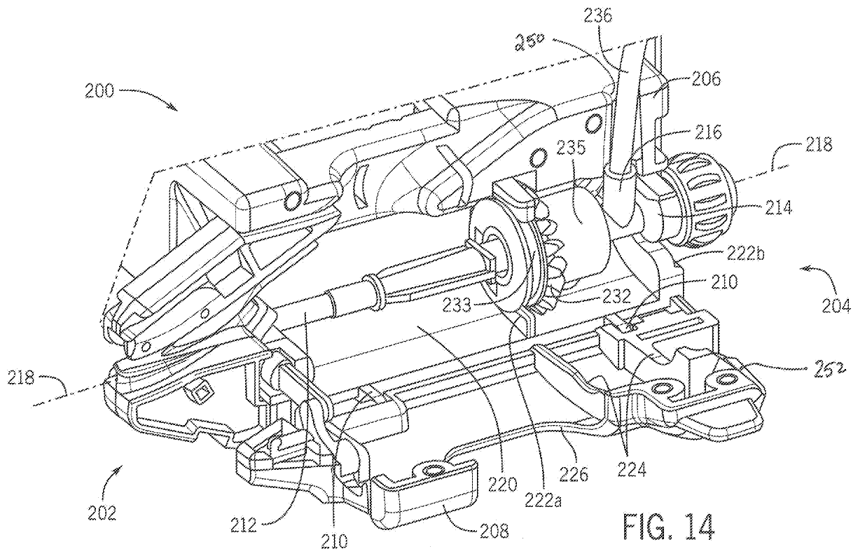

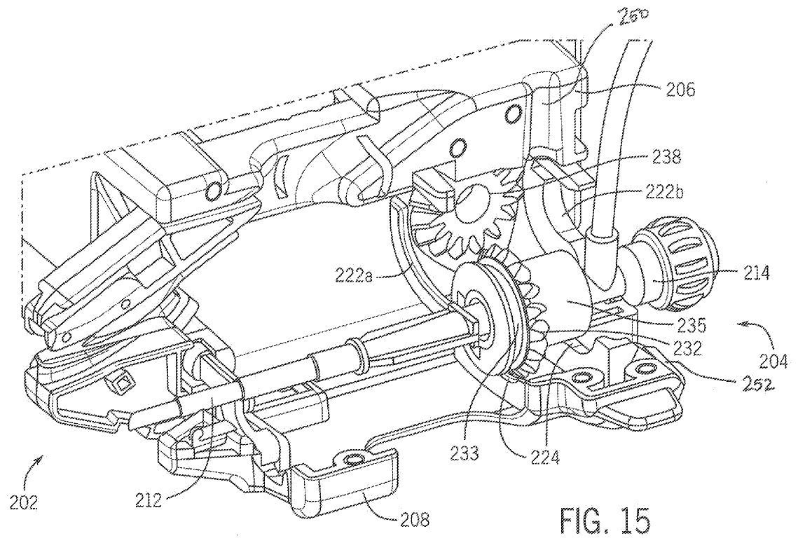

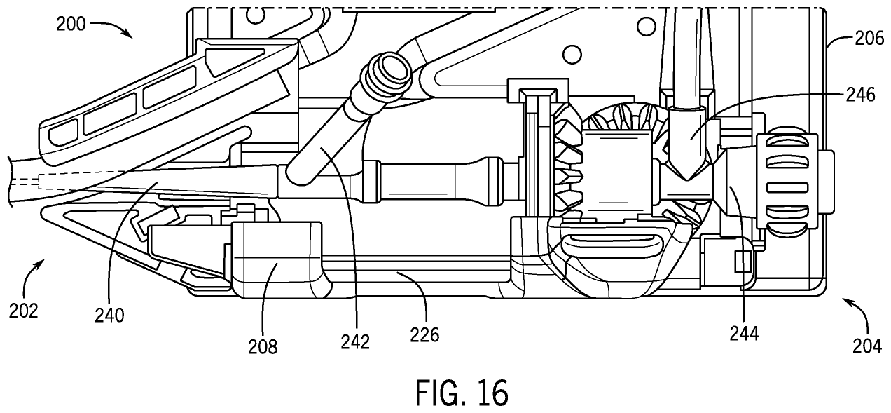

The cover 208 also includes a recess 224 in which an assembled EMD may be placed, for example, before the EMD is loaded into the cassette 200 as discussed further below with respect to FIG. 15. An opening 226 in the cover 208 enables use of an EMD with a port (e.g., a side port) in the cassette and allows access to the port of the EMD as discussed further below with respect to FIG. 16. The cassette housing 206 includes a recess 250 that is configured to receive a side port and/or a tube connected to the side port (e.g., side port 216 and tube 236 shown in FIG. 14) of a hemostasis valve positioned in the cassette housing 206. The cover 208 also includes a retaining element 252 that is complementary to the recess 250 and is configured to retain the side port when the cover 208 is in a closed position and to allow the tube 236 to be visible to a user when the cover is in a closed position as discussed further below with respect to FIG. 14. The cassette housing 206 includes a cradle 220 which is configured to receive an EMD (not shown) when the EMD is loaded into the cassette housing 206. A saddle 222a and a saddle 222b are located at a proximal end 204 of the cassette housing 206. In the embodiment shown in FIG. 12, the saddle 222a and the saddle 222b have a U-shape with a straight portion, namely, straight portion 227 for saddle 222a and straight portion 229 for saddle 222b. The saddle 222a is configured to receive and restrain a groove on a distal end of a hemostasis valve of an EMD and the saddle 222b is configured to receive and restrain a proximal end of the hemostasis valve of an EMD. For example, the saddle 222a and the saddle 222b may be configured to provide a snap fit for a groove and proximal end of a hemostasis valve placed in the saddles 222a and 222 as discussed further below. Cassette 200 also includes a bevel gear 238 that may be used to interface with a coupler of a drive module and to interface with an EMD to, for example, rotate the EMD.

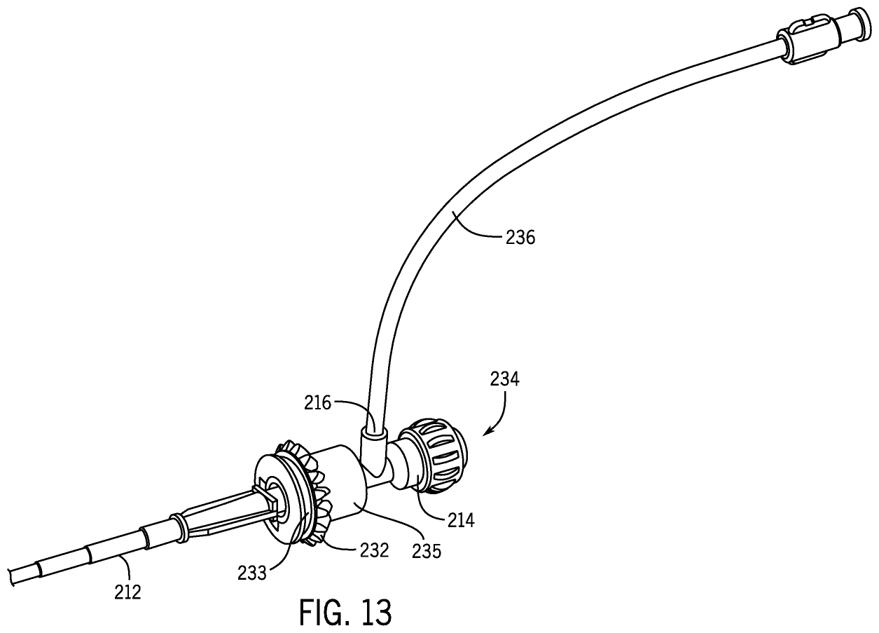

As mentioned, an EMD may be loaded into and positioned in the cassette 200. FIG. 13 is a perspective view of an example elongated medical device in accordance with an embodiment. The example EMD shown in FIG. 13 is a catheter 212. The catheter 212 is coupled to a hemostasis valve 214 (e.g., a rotating hemostasis valve) at a proximal end 234 of the EMD. A body 235 of the hemostasis valve includes a gear 232 and a groove 233 on a distal end of the body 235. The gear 232 is configured to interact with a gear (e.g., bevel gear 238 shown in FIG. 12) of a cassette. For example, when power is transferred from a drive module (e.g., via a coupler) on which the cassette is mounted to a gear (e.g., gear 238) in a cassette, the gear in the cassette interacts with the gear 232 on the catheter 212 to rotate the catheter 212. In addition, the body 235, gear 232 and groove 233 are configured to rotate while the proximal end 234 of the hemostasis valve 214 (including a side port 216) remains stationary. The side port 216 of the hemostasis valve 214, as mentioned above, may be connected to a tube 236 to facilitate the flow of a fluid (e.g., saline) to and from the hemostasis valve 214 and the catheter 212. When the EMD is loaded into a cassette (e.g., cassette 200 shown in FIG. 12) in a robotic drive (e.g., robotic drive 24 shown in FIG. 3), the tube 236 may be connected to a fluid source (not shown), for example, a pressurized bag.

FIG. 14 is a perspective view of a cassette with a cover in an open position and an elongated medical device in accordance with an embodiment. In FIG. 14, an EMD (e.g., the EMD shown in FIG. 13) is loaded into and positioned in a cassette 200. In particular, the EMD is positioned in a cradle 220 of the cassette housing 206. As mentioned above, the EMD may be a catheter 212 that is coupled to a hemostasis valve 214 with a side port 216, gear 232, groove 233 and body 235. The side port 216 is positioned in a vertical orientation so that the side port 216 points upward when the cassette 200 is vertically mounted on a drive module (not shown) in a robotic drive. Accordingly, a tube 236 connected to the side port 216 may be directed up and be draped over the top of the robotic drive. A recess 250 in the cassette housing 206 is configured to receive the side port 216 and the tube 236 coupled to the side port 216. When the cover 208 is in a closed position, a complimentary retaining element 252 retains the side port 216 in place during operation of the robotic drive, for example, the advancement and retraction of the device module that includes the cassette 200 and the rotation of the catheter 212. In an embodiment, the retaining element 252 is configured to allow all or a portion of the side tube 236 in the cassette housing 206 to be visible to a user in order to, for example, monitor the tube 236 for air bubbles. For example, the width of the retaining element 252 may be less than the width of side port 216 and the tube 236. In addition, the recess 250 of cassette housing 206 and the retaining element 252 of cover 208 may be configured to enable the orientation of the side port 216 in a desired direction (e.g., substantially vertical).

The groove 233 of the hemostasis valve 214 is positioned in the saddle 222a at the proximal end 204 of the cassette housing 204 and the proximal end of the hemostasis valve 214 is positioned in the saddle 222b at the proximal end 204 of the cassette housing 204. As mentioned above, the saddles 222a and 222b are configured to receive and restrain a hemostasis valve of an EMD. For example, the saddles 222b may be configured to provide a snap fit for a proximal end of hemostasis valve 214 placed in the saddle 222b. In an embodiment, the groove 233 of the hemostasis valve 214 may also be restrained by the saddle 222a, for example, with a snap fit. In an embodiment, the geometry of the groove compliments the geometry of the saddle 222a. The saddles 222a and 222b do not fully hold the groove 233 and the proximal end of the hemostasis valve 214, but is configured to sufficiently restrain (e.g., 90-90% located) the groove 233 and the proximal end of the hemostasis valve 214 so that the EMD does not fall out of the cassette when the cover 208 is opened or before the cover 208 is closed. When cover 208 is closed, the recess 224 provides the additional forces to keep the catheter 212, hemostasis valve 214 and gear 232 in place during operation of the robotic drive, for example, the advancement and retraction of the device module that includes the cassette 200 and the rotation of the catheter 212. Accordingly, the recess 224 is configured to complete the saddles 222a and 222b when the cover 208 is in a closed position. The cover 208 is also configured to push locate the groove 233 when the cover 208 is in the closed position (e.g., the closed position shown in FIG. 11). When in the closed position (e.g., as shown in FIG. 11), the cover 208 prevents a user or other elements of the system from coming into contact with the gear 232.

As mentioned above, the cover 208 of cassette 200 also includes a recess 224 in which an assembled EMD may be placed, for example, before the EMD is loaded into the cassette 200. FIG. 15 is a perspective view of a cassette with an elongated medical device positioned on a cover of the cassette in an open position before the elongated medical device is loaded in the cassette in accordance with an embodiment. In FIG. 15, the EMD is a catheter 212 that is coupled to a hemostasis valve 214 (including gear 232, groove 233 and body 235)). The catheter 212 and hemostasis valve 214 are placed in the recess 224 in the cover 208. Accordingly, the recess 224 may act as a shelf that temporarily lightly holds the assembled EMD, for example, before it is loaded into the cassette 200. In an embodiment, the groove 233 has a geometry complimentary to the recess 224 so that the groove 233 is meshed with and constrained by the recess 224 when resting on the cover 208, for example, the grove 233 may have a flange that locates on either side of the recess 224. As mentioned above, in an embodiment the cover 208 may include an opening 226. FIG. 16 is a front view of a cassette with a cover in an open position and an elongated medical device loaded in the cassette in accordance with an embodiment. In FIG. 16, the opening 226 in cover 208 may be used to allow the use of an EMD 240 that includes a side port 242. The opening 226 of cover 206 allows the cover 208 to be closed and still provide access to the side port 242. In addition, the opening 226 of cover 208 may be configured to enable the orientation of the side port 242 in a desired direction (e.g., outward, substantially vertical, etc.). The EMD in shown in FIG. 16 also coupled to a hemostasis valve 244 with a side port 246.

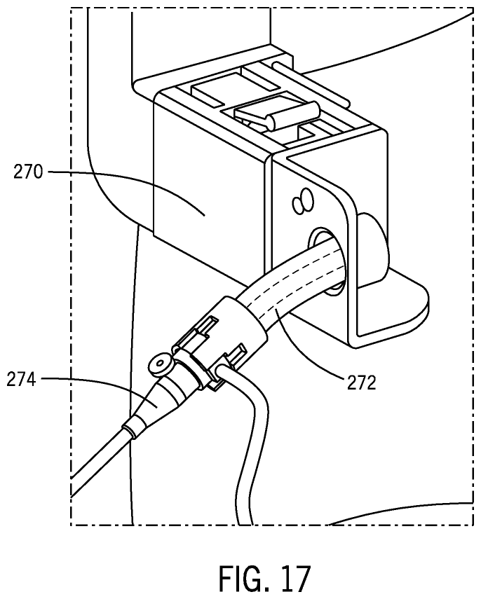

As mentioned above with respect to FIG. 3, the robotic drive 24 may also include a device support connection 72 connected to a device support 79a and a distal support arm 70. Device support connection 72 is used to provide support for a distal end of the device support 79a housed in the distal most device module 32a. The distal support arm 70 extends away from the robotic drive 24 and may be attached to a frame of the robotic drive 24, e.g. a frame of the linear member 60. A connector on the distal end of the device support 79a may be attached to the device support connection 72. In addition, an introducer interface support 74 may be connected to the device support connection 72 and an introducer sheath. FIG. 17 is a perspective view of an introducer interface support in accordance with an embodiment. In FIG. 17, an introducer interface support (or sheath connector) 272 is connected to a device support connection 270 and an introducer sheath 274. The introducer interface support 272 is configured to support an EMD (not shown) between the device support (e.g., device support 79a shown in FIG. 3) and the introducer sheath 274 connected to a distal end 276 of the introducer interface support 272. The introducer interface support 272 ensures that the EMD does not buckle or prolapse between the distal end of the device support (e.g., device support 79a show in FIG. 3) and the hub of the introducer sheath 274. The introducer interface support 272 is a flexible tube. The flexible tube of the introducer interface support 272 may be configured to provide the correct bend to help avoid misalignment and to account for a perturbation of the robotic drive or movement of the patient. In an embodiment, the introducer interface support 272 may also be used to redirect an EMD from a position that is axially aligned with the robotic drive and device axis to a position that is axially aligned with the introducer sheath 274. The introducer sheath 274 is inserted at an access point (e.g., the femoral artery) into a patient's vasculature that will least the EMD to the target location in the patient (e.g., a lesion). The introducer sheath 274 should be held in place so that it does not come out of the patient. In an embodiment, the device support connection 270 and the distal support arm 70 (shown in FIG. 3) may be used to fix the position of the introducer sheath 274 and may react forces on the introducer sheath 274 created from the friction between the introducer sheath 274 and the EMD moving inside the introducer sheath 274.

A control computing system as described herein may include a processor having a processing circuit. The processor may include a central purpose processor, application specific processors (ASICs), circuits containing one or more processing components, groups of distributed processing components, groups of distributed computers configured for processing, etc. configured to provide the functionality of module or subsystem components discussed herein. Memory units (e.g., memory device, storage device, etc.) are devices for storing data and/or computer code for completing and/or facilitating the various processes described in the present disclosure. Memory units may include volatile memory and/or non-volatile memory. Memory units may include database components, object code components, script components, and/or any other type of information structure for supporting the various activities described in the present disclosure. According to an exemplary embodiment, any distributed and/or local memory device of the past, present, or future may be utilized with the systems and methods of this disclosure. According to an exemplary embodiment, memory units are communicably connected to one or more associated processing circuit. This connection may be via a circuit or any other wired, wireless, or network connection and includes computer code for executing one or more processes described herein. A single memory unit may include a variety of individual memory devices, chips, disks, and/or other storage structures or systems. Module or subsystem components may be computer code (e.g., object code, program code, compiled code, script code, executable code, or any combination thereof) for conducting each module's respective functions.

This written description used examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims. The order and sequence of any process or method steps may be varied or re-sequenced according to alternative embodiments.

Many other changes and modifications may be made to the present invention without departing from the spirit thereof. The scope of these and other changes will become apparent from the appended claims.

We claim:

1. A cassette for use in a robotic drive of a catheter-based procedure system, the cassette comprising:
a housing comprising a cradle configured to receive an elongated medical device having a longitudinal device axis;
a connection mechanism coupled to the housing at a position below the longitudinal device axis; and
a cover pivotably coupled to the housing using the connection mechanism.

2. The apparatus according to claim 1, wherein the cover has a closed position and an open position and wherein when the cover is in the open position an axis of the cover between the connection mechanism and an outer edge of the cover is substantially perpendicular to the housing.

3. The cassette according to claim 1, wherein the connection mechanism is a hinge.

4. The cassette according to claim 1, further comprising a mechanical locking mechanism configured to hold the cover in the closed position.

5. The cassette according to claim 1, wherein the cover includes a recess configured to receive a hemostasis valve coupled to the elongated medical device when the cover is in the open position.

6. The cassette according to claim 1, wherein the cover includes an opening configured to receive a side port of the elongated medical device.

7. The cassette according to claim 5, wherein the opening is configured to orient the side port of the elongated medical device in a substantially vertical direction.

8. The cassette according to claim 1, wherein the housing further comprises a recess configured to receive a side port of a hemostasis valve coupled to the elongated medical device.

9. The cassette according to claim 8, wherein the cover includes a retaining element configured to orient the side port of the hemostasis valve in a substantially vertical direction.

10. A cassette for use in a robotic drive of a catheter-based procedure system, the cassette comprising:
a housing comprising a cradle configured to receive an elongated medical device having a longitudinal device axis, the housing having a distal end and a proximal end;
a saddle positioned on the proximal end of the housing, the saddle configured to receive and restrain a hemostasis valve coupled to the elongated medical device;
a connection mechanism coupled to the housing at a position below the longitudinal device axis; and
a cover pivotably coupled to the housing using the connection mechanism.

11. The cassette according to claim 10, wherein the saddle includes a snap feature to hold the hemostasis valve when the hemostasis valve is positioned in the saddle.

12. The cassette according to claim 10, wherein the saddle has a U-shape.

13. The cassette according to claim 10, wherein the cover has a closed position and an open position and wherein when the cover is in the open position an axis of the cover between the connection mechanism and an outer edge of the cover is substantially perpendicular to the housing.

14. The cassette according to claim 13, wherein the connection mechanism is a hinge.

15. The cassette according to claim 13, further comprising a magnet configured to hold the cover in the closed position.

16. The cassette according to claim 13, wherein the cover includes an opening configured to receive a side port of the elongated medical device.

17. The cassette according to claim 16, wherein the opening is configured to orient the side port of the elongated medical device in a substantially vertical direction.

18. The cassette according to claim 10, wherein the housing further comprises a recess configured to receive a side port of the hemostasis valve.

19. The cassette according to claim 18, wherein the cover includes a retaining element configured to orient the side port of the hemostasis valve in a substantially vertical direction.

20. A robotic drive system for driving one or more elongated medical devices, the robotic drive system comprising:
a linear member;
a device module coupled to the linear member;
a distal support arm having a device support connection located distal to the device module;
an introducer interface support coupled to the device support connection, the introducer interface support comprising a flexible tube; and
an introducer sheath coupled to the introducer interface support.
